(19) United States
(12) Patent Application Publication
(10) Pub. No.: US 2022/0351853 A1
(43) Pub. Date: Nov. 3, 2022

(54) ENVIRONMENT MANAGEMENT SYSTEM, ENVIRONMENT MANAGEMENT METHOD, AND NON-TRANSITORY COMPUTER READABLE MEDIUM STORING PROGRAM

(71) Applicant: NEC Corporation, Minato-ku, Tokyo (JP)

(72) Inventors: Yuji OHNO, Tokyo (JP); Toshinori HOSOI, Tokyo (JP); Masatake TAKAI, Tokyo (JP); Masahiro KUBO, Tokyo (JP); Shigemi KITAHARA, Tokyo (JP)

(73) Assignee: NEC Corporation, Minato-ku, Tokyo (JP)

(21) Appl. No.: 17/778,118

(22) PCT Filed: Sep. 14, 2020
(86) PCT No.: PCT/JP2020/034711

§ 371 Date: May 19, 2022

(30) Foreign Application Priority Data
Nov. 26, 2019 (JP) 2019-213530

(51) Int. Cl. G16H 40/67; G05B 13/04

(52) U.S. Cl. CPC G16H 40/67; G05B 13/042

(57) ABSTRACT

An environment management system, an environment management method, and a program that are capable of reducing the load imposed on a responder are provided. An environment management system includes: a determination unit configured to determine, based on a feature value of biological information of a target patient to be input, identification information indicating a condition of the target patient; an estimation unit configured to estimate environment control information for the target patient based on the identification information and an environment control prediction parameter that has been learned in advance; and an environment control unit configured to automatically change a surrounding environment of the target patient based on the environment control information for the target patient estimated by the estimation unit.

### TECHNICAL FIELD

The present invention relates to an environment management system, an environment management method, and a program.

### BACKGROUND ART

In hospitals and the like, nurses and care workers spend a large amount of their time dealing with patients regarding which there is risk of their displaying problematic behavior, such as falling from a bed, removing an intubation tube, making strange noises, or committing acts of violence, and thus are unable to focus on their primary care duties. Patients who display such problematic behavior are often in an acute state of confusion called “a restlessness (a restless state)”. Therefore, it is desirable that a restless state of a patient be detected before he/she displays problematic behavior and appropriate measures be taken.

For example, Patent Literature 1 discloses a biological information processing system comprising a determination unit configured to determine, based on features of input biological information of a target patient, discrimination information indicating whether or not a condition of the target patient has changed in comparison with a normal state, and an estimation unit configured to estimate countermeasure information for the target patient based on the discrimination information and countermeasure prediction parameters which are preliminarily learned.

### CITATION LIST

### Patent Literature

Patent Literature 1: International Patent Publication No. WO 2019/073927

### SUMMARY OF INVENTION

### Technical Problem

However, although the biological information processing system disclosed in Patent Literature 1 can detect a sign of a patient's restlessness from biological sensor information, estimate countermeasure information, and report it to a responder, such as a nurse, a care worker, and a therapist, the system cannot directly respond to the patient based on the countermeasure information. Therefore, there is a problem that it is necessary for the responder himself/herself to deal with the patient based on the countermeasure information and thus the load imposed on the responder has not been reduced.

The present invention has been made to solve the above-described problem and an object thereof is to provide an environment management system, an environment management method, and a program that are capable of reducing the load imposed on a responder.

### Solution to Problem

An environment management system according to a first example aspect of the present invention includes:

a determination unit configured to determine, based on a feature value of biological information of a target patient to be input, identification information indicating a condition of the target patient;

an estimation unit configured to estimate environment control information for the target patient based on the identification information and an environment control prediction parameter that has been learned in advance; and

an environment control unit configured to automatically change a surrounding environment of the target patient based on the environment control information for the target patient estimated by the estimation unit.

An environment management method according to a second example aspect of the present invention includes:

determining, based on a feature value of biological information of a target patient to be input, identification information indicating a condition of the target patient;

estimating environment control information for the target patient based on the identification information and an environment control prediction parameter that has been learned in advance; and

automatically changing a surrounding environment of the target patient based on the estimated environment control information for the target patient.

A program according to a third example aspect of the present invention causes a computer to execute:

processing of determining, based on a feature value of biological information of a target patient to be input, identification information indicating a condition of the target patient;

processing of estimating environment control information for the target patient based on the identification information and an environment control prediction parameter that has been learned in advance; and

processing of automatically changing a surrounding environment of the target patient based on the estimated environment control information for the target patient.

### Advantageous Effects of Invention

According to the present invention, it is possible to provide an environment management system, an environment management method, and a program that are capable of reducing the load imposed on a responder.

### BRIEF DESCRIPTION OF DRAWINGS

FIG. 1 is a block diagram showing a configuration of an environment management system according to a first example embodiment of the present invention;

FIG. 2 is a diagram showing an example of identification information;

FIG. 3 is a diagram showing an example of countermeasure information estimated by an environment control estimation unit;

FIG. 4 is a flowchart showing a flow of operations performed by an environment management system 100 shown in FIG. 1;

FIG. 5 is a block diagram showing a configuration of an environment management system according to a second example embodiment of the present invention;

FIG. 6 is a block diagram showing a configuration of a model generation unit according to the second example embodiment of the present invention;

FIG. 7 is a flowchart showing a flow of operations in which an environment management system 200 learns identification parameters and environment control prediction parameters;

FIG. 8 is a flowchart showing a flow of operations performed from when the environment management system 200 shown in FIG. 5 acquires biological information of a target patient to when it sends a notification about countermeasure information;

FIG. 9 is a block diagram showing a configuration of an environment management system according to a third example embodiment of the present invention;

FIG. 10 is a block diagram showing an example of a configuration of a communication apparatus; and

FIG. 11 is a block diagram showing an example of a hardware configuration of the environment management system.

### DESCRIPTION OF EMBODIMENTS

### First Example Embodiment

Example embodiments will be described hereinafter with reference to the drawings. Note that the same or the corresponding components/parts are denoted by the same reference symbols or numerals throughout the drawings, and the descriptions thereof will be omitted as appropriate.

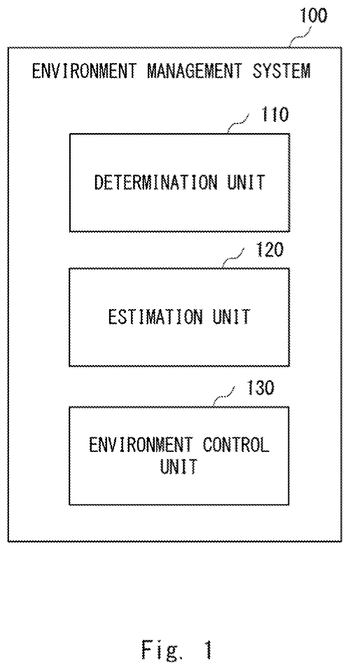

FIG. 1 is a block diagram showing a configuration of an environment management system according to a first example embodiment of the present invention. As shown in FIG. 1, an environment management system 100 includes a determination unit 110, an estimation unit (also referred to as an environment control estimation unit) 120, and an environment control unit 130. Although the environment management system is primarily used in hospitals, it may be used at home and in a nursing care facility during nursing or during caring. The hospitals include an acute-care hospital and a convalescent hospital. However, the present invention is not limited thereto.

The determination unit 110 receives a feature value related to biological information of a target patient and then determines identification information indicating a condition of the target patient based on the feature value. The biological information according to this example embodiment means information about a living body that can be measured by a sensor or the like. Specific examples of the biological information may include a heartbeat (pulse), respiration, a blood pressure, a core temperature, a level of consciousness, a skin temperature, a skin conductance response (Galvanic Skin Response (GSR)), a skin potential, a myoelectric potential, an electrocardiographic waveform, a waveform of a brain wave, an amount of sweating, a blood oxygen saturation, a waveform of a pulse wave, an optical functional brain mapping (Near-infrared Spectroscopy (NIRS)), a urine volume, and a pupil reflex. However, the present invention is not limited thereto. Such biological information may be acquired periodically (e.g., in the morning, at noon, and at night) from the target patient by a medical worker.

The feature value related to the biological information is information indicating a feature of the biological information generated by subjecting the biological information of a patient to calculation processing, and is, for example, temporal variation of a specific frequency band of the biological information. Specifically, the determination unit 110 can automatically determine whether the target patient is in a restless state or in a non-restless state as the condition of the target patient based on the feature value related to the biological information of the target patient. Note that the restless state (hereinafter may also be referred to as restlessness) means a state in which a target patient can display problematic behavior. Specifically, examples of the restless state include a state in which a target patient acts in an excessive manner and is restless, a state in which a target patient is not calm, and a state in which a target patient cannot control his/her mind so that it is normal. The magnitude of the restless state can be strong or weak, and the stronger the restless state, the more likely a patient is to display problematic behavior.

Further, the problematic behavior means behavior that prevents a patient from continuing appropriate treatment and rehabilitation, such as injuring himself/herself, injuring someone else, and imposing a load on nurses and the like. Specific examples of the problematic behavior include getting up on a bed, removing bed rails, leaving a bed, walking alone, wandering around, going to another floor of a hospital, falling out of a bed, falling over, playing with intravenous fluids or tubes, removing intravenous fluids or tubes, making strange noises, using abusive language, and committing acts of violence. However, behavior that corresponds to the problematic behavior differs depending on the state of patient. Note that the determination unit 110 may have a function of receiving biological information of a target patient and then calculating a feature value of the received biological information of the target patient. In this case, the determination unit 110 can calculate the feature value by performing smoothing processing, differential processing, or the like on the biological information. The determination unit 110 may, for example, include a plurality of bandpass filters having pass bands different from each other, differential filters, and the like, and may combine a plurality of values that can be obtained by performing, on the biological information, filter processing using a single filter, a plurality of combined filters, or the like, to thereby calculate the feature value.

In this example embodiment, identification information is information indicating whether or not a state of a target patient has changed from a normal state. For example, the identification information means information that includes a restlessness score indicating a possibility that a target patient is in a restless state. The restlessness score is calculated based on, for example, identification parameters that have been learned in advance and a feature value related to the biological information of the target patient. Note that the identification parameter means a parameter associating the feature value of the biological information with the restless state or the non-restless state. The aforementioned identification parameter can be generated, for example, by subjecting the feature value of the biological information obtained when the target patient is in the restless state and the feature value of the biological information obtained when the target patient is in the non-restless state to machine learning. The aforementioned identification parameter may be, for example, held in a storage device (not shown) disposed outside the environment management system 100. Further, when the determination unit 110 includes a storage unit (not shown), the storage unit of the determination unit 110 may hold the identification parameter. As described above, the identification parameter includes a parameter associating the feature value of the biological information with the restless state or the non-restless state. Therefore, by optimizing the identification parameter, it is possible to improve the accuracy of the identification information.

In this example embodiment, the restlessness score is an index indicating how restless a target patient is as compared to when he/she is in a normal state (a non-restless state). Specifically, the restlessness score can be expressed by, for example, a numerical value of zero or greater and one or less. In this case, for example, the target patient is more likely to be in a restless state or in a stronger restless state as the restlessness score becomes closer to one, and is more likely to be in a non-restless state as the restlessness score becomes closer to zero. Further, any number of values of zero or greater and one or less may be set as a threshold. In this case, the determination unit 110 may determine whether the target patient is in a restless state or a non-restless state based on whether or not the restlessness score of the target patient exceeds the threshold. Further, the restlessness score may be expressed by, for example, two values of zero and one. Specifically, for example, the determination unit 110 may output zero when the restlessness score is less than the threshold, while it may output one when the restlessness score is equal to or greater than the threshold. In this case, for example, it may be assumed that the target patient is in a restless state when the restlessness score is one, while the target patient is in a non-restless state when the restlessness score is zero. Further, a plurality of thresholds may be used to classify the levels of a restless state. For example, in a case in which two thresholds are used, when it is assumed that a threshold 2 is greater than a threshold 1, a level two may be set when the restlessness score is equal to or greater than the threshold 2, a level one may be set when the restlessness score is equal to or greater than the threshold 1 but less than the threshold 2, and a level zero may be set when the restlessness score is less than a threshold 0. In this case, it is determined that the higher the level, the stronger the restless state. The determination unit 110 can automatically determine identification information (a restlessness score) of a target patient based on, for example, a feature value of input biological information of the target patient and identification parameters received from the outside. Note that a threshold may be changed sequentially in accordance with a state of a patient and a time period.

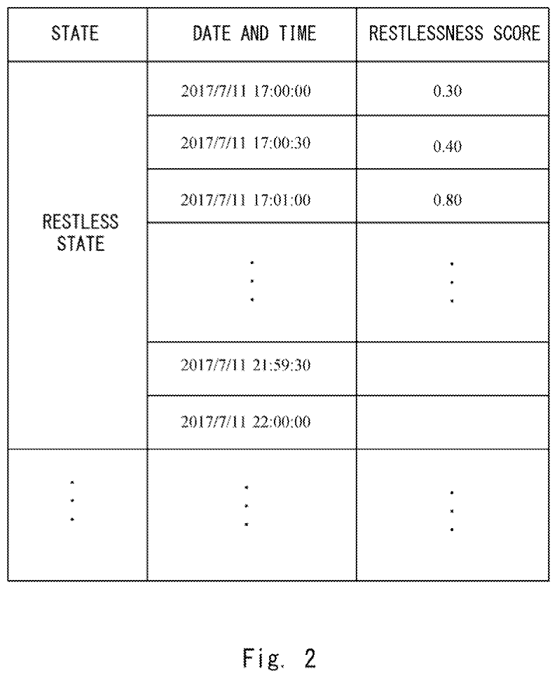

FIG. 2 is a diagram showing an example of identification information. As shown in FIG. 2, the identification information includes at least a state of a target patient, the date and time when biological information was measured, and a restlessness score. Specifically, for example, the identification information shown in FIG. 2 indicates that the restlessness score of the target patient at “17:01:00 on Jul. 11, 2017” is “0.80”, and that the state of the target patient becomes a restless state soon or the target patient is already in a restless state. Note that the identification information shown in FIG. 2 includes the state and the restlessness score of the target patient at 30-second intervals, but this is merely an example and is not intended to limit a measurement interval of biological information or the date and time when a restlessness score is calculated.

The environment control estimation unit 120 estimates countermeasure information including at least an environment control method to be applied to the target patient and a countermeasure score indicating the degree of an effect of the environment control method based on the identification information (the restlessness score) determined by the determination unit 110 and environment control prediction parameters that have been learned in advance.

Note that environment control means to change a surrounding environment of a target patient. The aforementioned surrounding environment is an environment that can affect a target patient, and may be, for example, a private room where the patient is present. When the room is a room shared with a plurality of patients, the surrounding environment may be a shared room or a space separated by a curtain. Further, the environment control preferably stimulates one of the sensory organs of a patient. Examples of the sensory organs include, in the large classification, organs related to somatic sensation, visceral sensation, special sensation, a sense of equilibrium, proprioceptive sensation, and itching sensation. Further, examples of the sensory organs include, in the small classification, organs related to visual sensation, auditory sensation, olfactory sensation, gustatory sensation, tactile sensation, a sense of equilibrium, and turning sensation.

Examples of the above environment control include projecting a specific video image onto a display, playing specific music from a speaker, emitting a specific scent from a diffuser, having a patient talk with a specific person (e.g., a family member and a responder) and a virtual person (e.g., idle Artificial Intelligence (hereinafter referred to as AI), family AI, and responder AI) via an environment management system, changing a condition of a bed (the angle of the bed or the sheet thereof), providing vibration or electrical stimulation to a patient, moving a bed to a specific location, changing a temperature or humidity, controlling a lighting apparatus (changing its brightness and color temperature), and automatically opening and closing a curtain. The display may be a television set located in an area around the bed, or a projector may be used to project a video image onto a surface near the bed such as the ceiling. When specific music is played from a speaker, in consideration of the influence of the music on the surrounding patients, a sound reproduction technique for producing a directional sound may be used for the speaker so that only a target patient can hear the sound. For example, a speaker built into a pillow of a target patient or a speaker near the pillow may be used. Further, the sound may be reproduced by combining a plurality of ultrasonic waves. When the lighting apparatus is controlled, the color temperature may be changed to a high color temperature (daytime white or daylight color) during the daytime period and a low color temperature (light bulb color) during the nighttime period. The color may be changed so that it is bright during the daytime and it is dark during the nighttime.

Further, the environment control prediction parameter means a parameter associating the environment control method applied to a target patient in a restless state in the past with the variation of the feature value related to the biological information or the variation of the restlessness score in a predetermined period of time before and after the environment control method is applied. The above environment control prediction parameter can be generated, for example, by subjecting, to machine learning, the variation of the feature value of the biological information or the variation of the restlessness score due to the application of the environment control method. That is, the environment control estimation unit 120 can estimate, based on past cases, countermeasure information that uses environment control in accordance with the variation of the feature value of the biological information or the variation of the restlessness score over a predetermined period of time. Specifically, for example, the environment control estimation unit 120 can estimate the countermeasure information that uses environment control at “22:00:00 on Jul. 11, 2017” in accordance with the restlessness score in the time period of “17:00:00 on Jul. 11, 2017” to “22:00:00 on Jul. 11, 2017” based on the restlessness score in the above time period and environment control prediction parameters. In this case, for example, the environment control prediction parameters learned between “0:00:00 on Jul. 1, 2017” and “23:59:59 on Jul. 10, 2017” may be used. By doing so, the environment control estimation unit 120 can also estimate, for example, that even when a target patient is in a normal state (a non-restless state) at the point in time of “22:00:00 on Jul. 11, 2017”, the state of the target patient can soon change to a non-normal state (a restless state). In this case, the environment control estimation unit 120 can estimate countermeasure information including a countermeasure that can suppress the change of the state of the target patient from the normal state (the non-restless state) to the non-normal state (the restless state).

Note that the environment control estimation unit 120 may estimate the countermeasure information while taking additional information of the identification information into account. In this example embodiment, the additional information means information that affects the identification information of the target patient. Specific examples of the additional information include the surroundings of a target patient and the influence of applying a countermeasure to the target patient on the surroundings (surrounding influence information), the magnitude of the load imposed on the patient (a patient load), the magnitude of the load imposed on nurses or the like (a responder load), the amount of time required to apply the countermeasure to the target patient, the financial cost of applying the countermeasure, and information included in a medical record (an electronic medical record). In this case, by taking the additional information into account, the environment control estimation unit 120 can estimate the countermeasure information in which the load imposed on the target patient, nurses, etc., the influence of applying the countermeasure to the target patient on the surrounding patients, and the like are taken into account. That is, the environment control estimation unit 120 estimates the identification information by taking the additional information into account, whereby the accuracy of the identification information is improved, and the load imposed on the target patient, nurses, etc. is further reduced.

The surrounding influence information is information including the degree of influence of applying an environment control method to a target patient on the surroundings, such as causing trouble to patients surrounding the target patient. Specifically, the surrounding influence information means, for example, information indicating whether the target patient's room is a private room (i.e., there are no other patients in the room) or a shared room (there are other patients in the room), whether the curtains are closed or open in a case of the shared room, whether or not the time to apply a countermeasure is daytime, whether or not it is necessary to brighten the room when the countermeasure is applied after the lights have been turned off, and whether or not sounds which the surrounding patients can hear are generated when the countermeasure is applied after the lights have been turned off. The surrounding influence means an influence such as waking up the surrounding patients who are sleeping, making them unable to sleep, and making them angry because it is noisy. Note that the above-described surrounding influence information is merely an example, and is not intended to limit the present invention.

The patient load means a load imposed on the body of a target patient by applying a countermeasure thereto. For example, the patient load is increased when a countermeasure is applied in which the target patient is made to have a conversation with a real person or a virtual person, while the patient load is reduced when a countermeasure is applied in which low-tempo music is played from a speaker near the target patient.

The information included in an electronic medical record means, for example, information about age, gender, height, weight, a family structure, a personality, hobbies, preferences, the presence or absence of experience of complications, a medication history, blood components, a medical history, excretion, and eating and drinking. Note that the above-described information included in an electronic medical record is merely an example, and other pieces of information may be included in the electronic medical record.

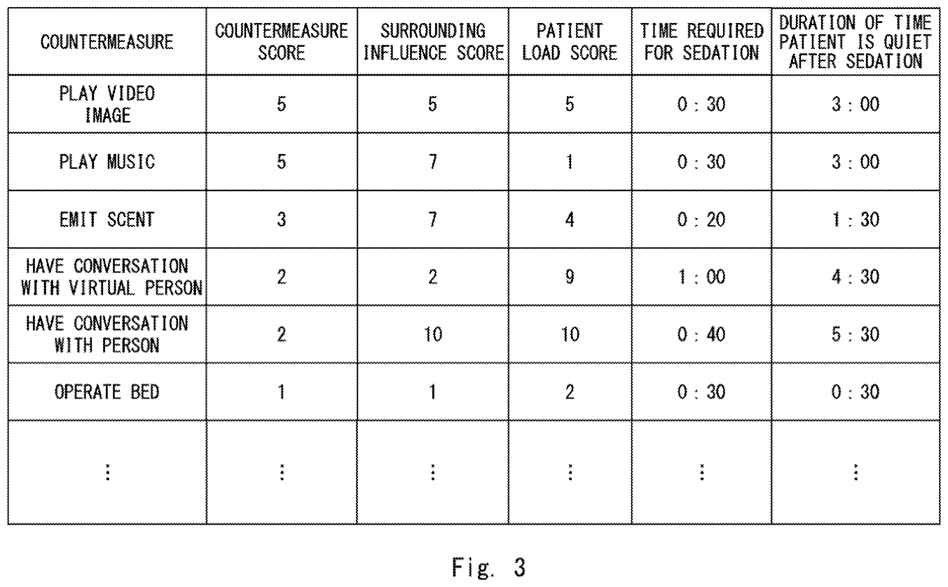

FIG. 3 is a diagram showing an example of countermeasure information estimated by the environment control estimation unit 120. As shown in FIG. 3, the countermeasure information includes, for example, an environment control method, a countermeasure score, a surrounding influence score, a patient load score, a time required for sedation, and a duration of time during which the patient is quiet after the sedation. The surrounding influence score and the patient load score are pieces of information about additional information, and a time required for sedation and a duration of time during which the patient is quiet after the sedation are pieces of information about identification information. Note that although the countermeasure information shown in FIG. 3 includes two types of additional information, i.e., the surrounding influence score and the patient load score, this is merely an example, and the countermeasure information may further include a plurality of pieces of additional information or may not include additional information. The scores shown in FIG. 3 are merely examples and are not limited thereto. Further, the score may be changed sequentially. For example, the score may vary between daytime and nighttime, or may vary depending on whether a patient is in a private or shared room.

The environment control method means information indicating a type of a treatment for performing environment control on a target patient, for example, information about a treatment that does not allow the target patient to cause a problem or that suppresses a non-normal state (a restless state) of the target patient. Specifically, the countermeasures are classified into the countermeasures mainly applied during the nighttime and the countermeasures mainly applied during the daytime. Examples of the countermeasures mainly applied during the nighttime may include moving a bed, providing vibration or electrical stimulation to a patient's body, changing a temperature or humidity, adjusting a patient's body temperature, circulating wind (e.g., turn on a fan and/or an air conditioner), showing a video image in which objects do not move greatly, having a patient listen to low-tempo music, having a patient smell something (smell an aroma), having a patient talk with a virtual person, darkening illumination, and lowering the color temperature of illumination. Further, examples of the countermeasures mainly applied during the daytime may include having a patient have a conversation with a real person (e.g., a family member or a nurse) via videophone or the like, having a patient talk with a virtual person, adjusting illumination of a room, adjusting a patient's body temperature, adjusting a room temperature (e.g., turn on a fan and/or an air conditioner), showing a specific video image (e.g., a television program), having a patient listen to music, having a patient smell something (smell an aroma), brightening illumination, and increasing the color temperature of illumination. The above-described countermeasures are merely examples and are not intended to limit the present invention.

Note that since the movement (the time change) of the restlessness score shown in FIG. 2 is different for each target patient, the environment control estimation unit 120 can estimate different countermeasure information for each patient even when the values of the restlessness scores for patients at a certain timing are the same. Further, the environment control estimation unit 120 may estimate different countermeasures in accordance with the magnitude of the restlessness score. Specifically, for example, the environment control estimation unit 120 can estimate mainly a countermeasure having a large sedative effect when the restlessness score is relatively large, and estimate mainly a countermeasure in which a small load is imposed on the body of a target patient having a weak body even when the restlessness score is large.

The countermeasure score means a value that indicates the effectiveness of a countermeasure applied to a target patient. The countermeasure score is expressed, for example, in five stages of 1 to 5, in which the higher the number, the more effective the countermeasure. Specifically, the countermeasure information shown in FIG. 3 indicates that, for a target patient, showing specific video images (e.g., video images of his/her home and family, video images of his/her hobbies, video images of his/her memories, video images of his/her travels, his/her favorite scenery, and video images of his/her workplace) and having the target patient listen to specific music are highly effective, while moving the bed is less effective. Further, the countermeasure score may be expressed in more than five stages or less than five stages. That is, the countermeasure scores are associated with the respective countermeasures included in the countermeasure information according to this example embodiment. By doing the above, in this example embodiment, the degree and the accuracy of the effect of the countermeasure included in the countermeasure information become apparent.

The surrounding influence score means a value that indicates the influence of applying a countermeasure to a target patient on the surrounding environment. The surrounding influence score is expressed, for example, in 10 stages of 1 to 10, in which the higher the number, the smaller the influence of applying a countermeasure to a target patient on the surroundings. Specifically, the countermeasure information shown in FIG. 3 indicates that, for the target patient, moving the bed has a small influence on the surrounding environment, while showing specific video images (e.g., showing television programs) has a large influence on the surrounding environment if he/she is in a shared room since the television set emits light and sound. Note that the surrounding influence score may be expressed in more than 10 stages or less than 10 stages. The surrounding influence score may be expressed in other forms.

The patient load score means a value that indicates the magnitude of the load imposed on a target patient by applying a countermeasure to the target patient. The patient load score is, for example, a score expressed in 10 stages of 1 to 10, in which the higher the number, the greater the load imposed on a patient. Specifically, the countermeasure information shown in FIG. 3 indicates that talking with a person imposes a large load on the patient, while playing music imposes a small load on the patient. The countermeasure information other than the above ones may indicate that administering a sleeping analgesic to a target patient and having a target patient wear a restraint impose a large load on the patient, while continuously talking to a target patient imposes a small load on the patient. Note that the patient load score may be expressed in more than 10 stages or less than 10 stages. The patient load score may be expressed in other forms.

Note that, when information other than the surrounding influence score and the patient load score is included in the countermeasure information as additional information, the score of the additional information, for example, may be evaluated in the stages of 1 to 10 in a way similar to that by which the surrounding influence score and the patient load score are evaluated, and the evaluated additional information may be included in the countermeasure information.

The time required for sedation is an estimated time period required for the state of a target patient to change from a restless state to a non-restless state when a countermeasure is applied to the target patient. Specifically, FIG. 3 shows that when a specific video image is shown to a target patient who is in the restless state, the state of the target patient changes from the restless state to the non-restless state 30 minutes after the viewing of the video image. The change from the restless state to the non-restless state can be determined, for example, by the fact that the restlessness score has been reduced from a value equal to or greater than a threshold to a value less than the threshold.

The duration of time during which the patient is quiet after the sedation is an estimated time period during which the non-restless state of a target patient continues after his/her state changes from the restless state to the non-restless state. Specifically, FIG. 3 shows that the non-restless state of the target patient continues for three hours when a specific video image is shown to the target patient. The duration of the non-restless state can be determined, for example, from the time that the restlessness score continues to be below the threshold.

The environment control unit 130 automatically changes an environment around a target patient based on countermeasure information for the target patient estimated by the environment control estimation unit 120. The environment around the target patient may include various types of apparatuses disposed in an entire private room when the target patient is in the private room, and may include various types of apparatuses disposed in an entire shared room or in a space inside the curtain surrounding the bed of the target patient when he/she is in the shared room. Examples of the various types of apparatuses include, but are not limited to, an air conditioner, a fan, a heater, a luminaire, a speaker, a music reproduction apparatus, a television set, a projector, an electric reclining bed, a massage machine (a pillow type, a body pillow type, etc.), a smartphone, a smart speaker, an aroma diffuser, a smartwatch, a game device, a land-line telephone, a personal computer (PC), an electric curtain, and an electric bed.

The various types of apparatuses are connected to a control apparatus that controls the operations of the various types of apparatuses through a network. Examples of the network here include a local area network (LAN), and a wide area network (WAN) such as the Internet. Further, for example, the communication network can be implemented by using any known network protocol including various types of wired or wireless protocols, such as Ethernet, Universal Serial Bus (USB), FIREWIRE (registered trademark), Global System for Mobile Communications (GSM (registered trademark)), Enhanced Data GSM (registered trademark) Environment (EDGE), code division multiple access (CDMA), time division multiple access (TDMA), Bluetooth (registered trademark), Wi-Fi (registered trademark), voice over Internet Protocol (VoIP), Wi-MAX (registered trademark), or any other suitable communication protocol.

### [Operation of Environment Management System 100]

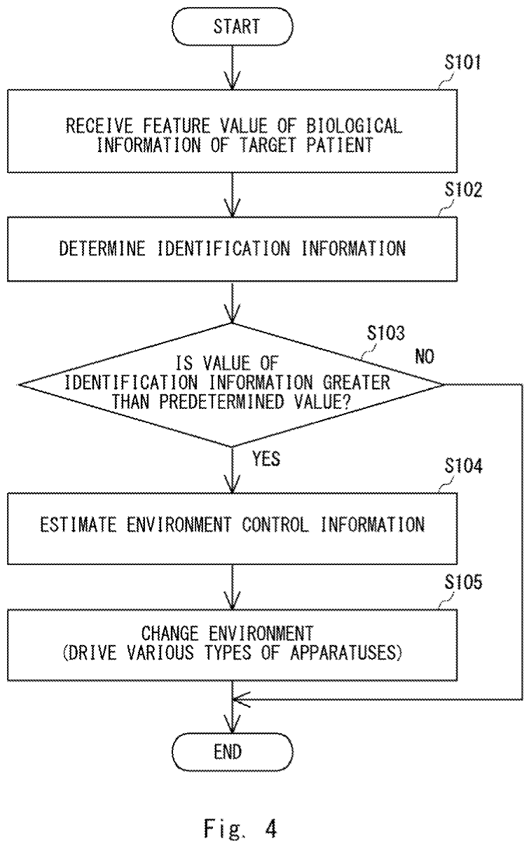

FIG. 4 is a flowchart showing a flow of operations performed by the environment management system 100 shown in FIG. 1. The flow of operations performed by the environment management system 100 will be described below with reference to FIGS. 1 and 4.

First, the determination unit 110 receives a feature value related to biological information of a target patient from the outside (Step S101).

Next, the determination unit 110 determines identification information indicating whether the target patient is restless or non-restless based on the feature value related to the biological information and identification parameters (Step S102).

Next, if the value (the restlessness score) of the identification information is less than a predetermined value (“NO” in Step S103), the environment management system 100 ends the operation. Note that when the operation of the environment management system 100 is ended, the various types of apparatuses may not only be stopped, but may also be returned to their original states as appropriate. For example, when the bed where the patient sleeps is raised, the bed may be returned to its original position and then the operation of the environment management system 100 may be ended.

On the other hand, if the value of the identification information is equal to or greater than the predetermined value (“YES” in Step S103), the environment control estimation unit 120 estimates countermeasure information that uses environment control corresponding to the identification information (Step S104).

The environment control unit 130 automatically changes the environment around the target patient based on the countermeasure information for the target patient estimated by the environment control estimation unit 120 (Step S105).

As described above, the environment management system 100 according to this example embodiment can estimate the countermeasure information shown in FIG. 3 based on past cases. Therefore, the environment management system 100 can apply an optimum environment control method to the target patient by taking the countermeasure information shown in FIG. 3 into account. By doing the above, this example embodiment can reduce the load imposed on a responder such as a nurse. Further, in this example embodiment, the environment management system 100 can predict that the state of a target patient is to be changed to a restless state while he/she is in a non-restless state, and can apply a countermeasure to the target patient before he/she displays problematic behavior. By doing the above, in this example embodiment, it is possible to suppress the change of the state of the target patient from a non-restless state to a restless state or the target patient's display of problematic behavior. Further, the environment management system 100 can estimate the countermeasure information while taking into account the influence of applying a countermeasure to the target patient on the surrounding environment. Therefore, when there are a plurality of countermeasures having almost the same effect that can be applied to the target patient, the environment management system 100 can avoid applying, for example, a countermeasure that causes trouble to the surrounding patients. By doing the above, this example embodiment can also suppress the influence of applying the countermeasure to the patient on the surrounding environment.

### Second Example Embodiment

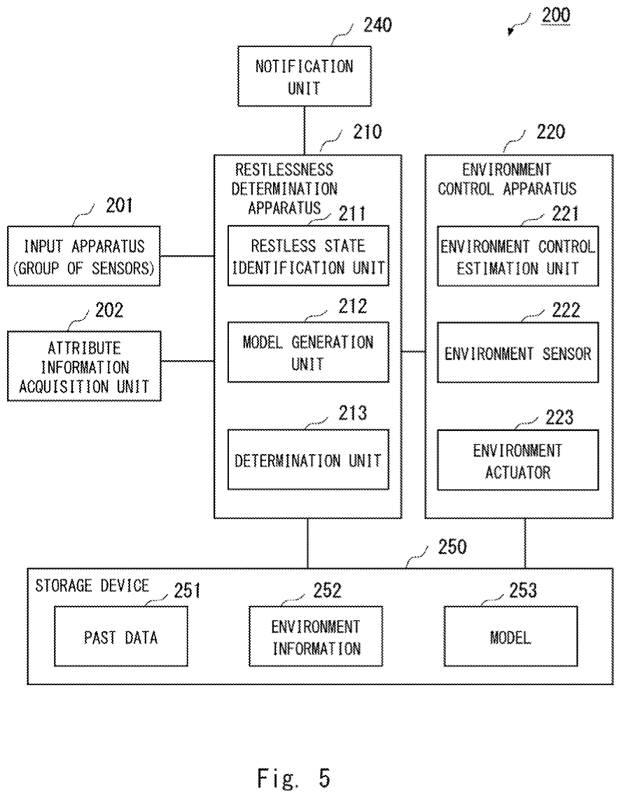

FIG. 5 is a block diagram showing a configuration of an environment management system according to a second example embodiment of the present invention. As shown in FIG. 5, an environment management system 200 includes an input apparatus 201, an attribute information acquisition unit 202, a restlessness determination apparatus 210, an environment control apparatus 220, a notification unit 240, and a storage device 250. Further, the restlessness determination apparatus 210 includes a determination unit 213, a model generation unit 212, and a restless state identification unit 211. Further, the environment control apparatus 220 includes an environment control estimation unit 221, an environment sensor 222, and an environment actuator 223.

The input apparatus 201 is a group of sensors for acquiring biological information of a target patient, and may be, for example, a contact type sensor such as a wearable device, a wristwatch type sensor (e.g., a smartwatch), and a bed type sensor, or a non-contact type sensor such as an infrared type sensor, a radio wave type sensor, and a camera that captures images of the target patient. It is desirable for the input apparatus 201 to observe the state of the target patient and continuously acquire time series data thereof. The term “continuously” includes not only a case when a patient wears a wearable device and the device obtains biological information at all times, but also a case when the patient is in a private room or a space inside the curtain where the patient's bed is located and a camera or the like obtains biological information at all times and a case when biological information is not obtained since the patient is in a bathroom or the like.

The attribute information acquisition unit 202 acquires attribute information of a target patient from, for example, an electronic medical record. The attribute information includes, but is not limited to, information such as the age, gender, career, home, family, hobbies, memories, favorite scenery, workplace, favorite music, favorite scent, favorite idol, close friends, and the like of the target patient.

The restlessness determination apparatus 210 determines the restless state of the target patient based on information obtained from the input apparatus 201 and the attribute information acquisition unit 202. The restless state identification unit 211 receives biological information of the target patient detected by the input apparatus 201 such as a biological sensor and then calculates a restlessness score from a feature value related to the received biological information and a generated model (identification parameters held by the storage device 250). Further, the restless state identification unit 211 may have a function of storing the determined identification information in the storage unit 250. The model generation unit 213 generates a model from the aforementioned feature values in a restless state and the feature values in a non-restless state acquired in advance. Further, the determination unit 213 determines whether the target patient is restless or non-restless from the identification information (the restlessness score).

The storage device 250 includes at least past data 251, environment information 252, and a model 253. The past data 251 can include feature values of a plurality of pieces of past biological information, past countermeasure information using environment control, and past identification information. The environment information 252 can include time series data of the temperature, humidity, brightness, and volume of air of the environment of the target patient, location information of the patient, and the like. The model 253 can include the identification parameters and countermeasure prediction parameters using environment control.

The environment control apparatus 220 automatically changes the surrounding environment of the target patient appropriately so that the state of the target patient becomes a normal state (a non-restless state) when the determination unit 213 (the restlessness determination apparatus 210) determines that the target patient is in a restless state.

The environment control estimation unit 221 estimates countermeasure information that uses environment control based on the identification information determined by the determination unit 213 (the restlessness determination apparatus 210) and the countermeasure prediction parameters held by the storage device 250. Further, the environment control estimation unit 221 may have a function of storing the estimated countermeasure information in the storage unit 250. Note that the environment control estimation unit 221 according to this example embodiment may estimate countermeasure information other than that using environment control. The countermeasure information other than that using environment control usually indicates a countermeasure that requires some kind of help from a responder such as a nurse, and examples of this countermeasure information include administering a painkiller, having a patient drink water, and taking a patient to a toilet.

The environment sensor 222 acquires information about the surrounding environment of the target patient, and examples of this sensor can include a temperature sensor, a humidity sensor, an illuminance sensor, a camera, a microphone, a bed type sensor, and an infrared type sensor.

The environment actuator 223 operates various types of appropriate apparatuses (e.g., an air conditioner, illumination, a speaker, a television set, and a projector) based on the countermeasure information using environment control estimated by the environment control estimation unit 221. By doing the above, it is possible to appropriately change the surrounding environment of the target patient without a responder such as a nurse.

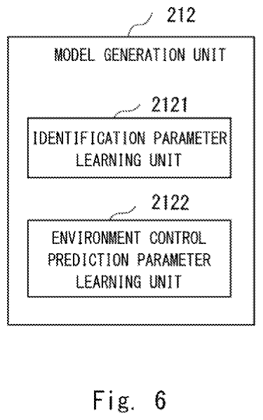

The model generation unit 212 can learn the identification parameters and environment control prediction parameters by machine learning. Specifically, as shown in FIG. 6, the model generation unit 212 includes an identification parameter learning unit 2121 and an environment control prediction parameter learning unit 2122.

The identification parameter learning unit 2121 learns the relationship between the feature values of the plurality of pieces of past biological information and whether the target patient is in the restless state or the non-restless state, thereby learning the identification parameters. Further, the identification parameter learning unit 2121 can store the generated identification parameters in the storage device 250.

The environment control prediction parameter learning unit 2122 learns environment control prediction parameters based on a plurality of countermeasures to be applied when a plurality of patients including a target patient are all in the restless state and a plurality of feature values related to pieces of biological information of the plurality of respective patients in a predetermined period of time. Further, the environment control prediction parameter learning unit 2122 can store the generated environment control prediction parameters in the storage device 250. In this example embodiment, the environment management system 200 has a function of learning environment control prediction parameters. Therefore, in this example embodiment, it is possible to improve the accuracy of countermeasure information by repeating the learning of environment control prediction parameters.

The notification unit 240 notifies a nurse or the like about countermeasure information other than that using environment control together with countermeasure information using environment control estimated by the environment control estimation unit 221. For example, the notification unit 240 is configured so that after the environment control estimation unit 221 estimates countermeasure information using environment control, it automatically notifies a nurse or the like about the countermeasure information by voice or a video image. Further, the notification unit 240 is configured to notify a responder such as a nurse about countermeasure information (e.g., having a patient take medicine) other than that using environment control. The notification unit 240 can also send, for example, an instruction to urge a nurse or the like to place an order for medicine. The above-described notification unit 240 may be composed of, for example, a general speaker or a general display. By doing the above, a nurse or the like can easily know countermeasure information using environment control for a target patient by a notification sent from the notification unit 240. Further, the notification unit 240 may notify a portable terminal (e.g., a smartphone and a tablet) or a wearable terminal about countermeasure information, which portable and wearable terminal is in the possession of a nurse or the like and is capable of enabling the environment management system 200 (the notification unit 240) to be communicated with. By doing the above, since the countermeasure information other than that using environment control is notified from the notification unit 240, a nurse or the like can check the estimated countermeasure information anywhere. Thus, the nurse or the like can also appropriately deal with a target patient based on the countermeasure information other than that using environment control.

### [Learning Operation]

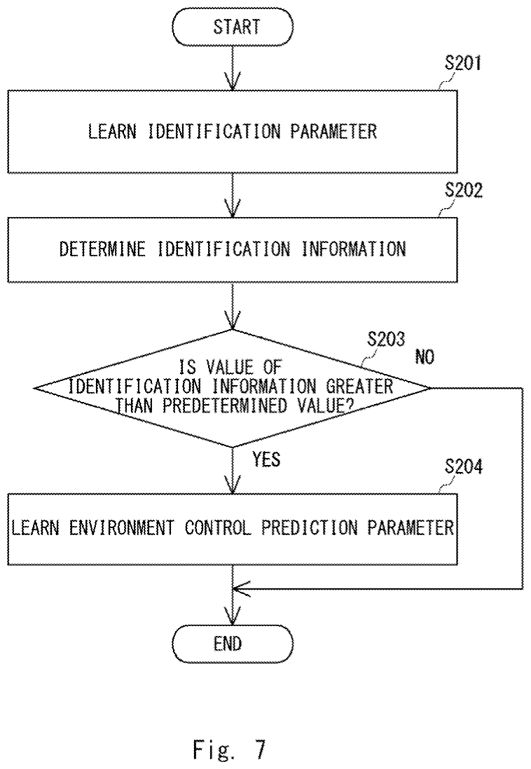

Next, a flow of operations in which the environment management system 200 learns identification parameters and environment control prediction parameters will be described with reference to FIGS. 5, 6, and 7. FIG. 7 is a flowchart showing the flow of operations in which the environment management system 200 learns identification parameters and environment control prediction parameters.

First, the identification parameter learning unit 2121 learns identification parameters (Step S201). Specifically, the identification parameter learning unit 2121 learns, by machine learning, the identification parameters by using the feature value calculated from biological information measured in advance when a patient is in a restless state and the feature value calculated from biological information measured in advance when a patient is in a non-restless state as training data. The biological information for learning identification parameters may be biological information of a target patient or biological information of an unspecified patient.

Next, the determination unit 211 determines identification information indicating whether a target patient is in a “restless state” or a “non-restless state” based on the measured biological information of the target patient and the identification parameters (Step S202).

Next, in Step S203, if the value of the identification information is less than a predetermined value (“NO” in Step S203), the environment management system 200 ends the learning operation since a nurse or the like does not apply a countermeasure to the target patient.

On the other hand, if the value of the identification information is equal to or greater than the predetermined value in Step S203 (“YES” in Step S203), the environment control prediction parameter learning unit 2122 learns environment control prediction parameters (Step S204). Specifically, the environment control prediction parameter learning unit 2122 learns, by machine learning, the environment control prediction parameters by using the relationship between the environment control method applied to the target patient and the temporal variation of the identification information of the target patient due to the application of the countermeasure.

The environment control prediction parameter learning unit 2122 learns the environment control prediction parameters by using a large amount of training data. Note that, since the environment control prediction parameter learning unit 2122 uses machine learning, the accuracy of the environment control prediction parameter improves as the amount of data to be learned increases. That is, the restless state of a target patient can be suppressed more efficiently, and thus the load imposed on a responder can be reduced.

### [Operation of Environment Management System 200]

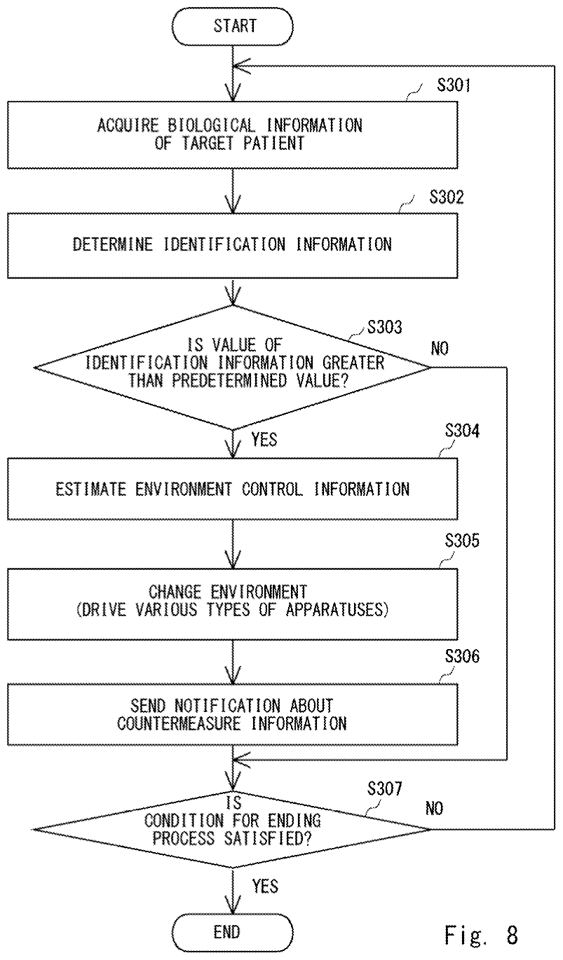

FIG. 8 is a flowchart showing a flow of operations performed from when the environment management system 200 shown in FIG. 5 acquires biological information of a target patient to when it sends a notification about countermeasure information. The flow of operations performed by the environment management system 200 will be described below with reference to FIGS. 5 and 8.

First, the restlessness determination apparatus 210 (the determination unit 213) receives biological information of a target patient measured by a biological sensor or the like and then calculates a feature value related to the received biological information (Step S301). At this time, the determination unit 213 may acquire a feature value related to the biological information of the target patient from the outside.

Next, the restlessness determination apparatus 210 (the determination unit 213) determines identification information indicating whether the target patient is in a “restless state” or a “non-restless state” based on the calculated feature value and identification parameters held by the storage device 250 (Step S302).

Next, if the value of the identification information is equal to or greater than a predetermined value (“YES” in Step S303), the environment control estimation unit 221 estimates, based on the identification information and environment control prediction parameters held by a storage unit 140, countermeasure information including at least one piece of countermeasure information other than that using environment control in addition to an environment control method to be applied to the target patient (Step S304).

Next, the environment actuator 223 of the environment control apparatus 220 operates various types of apparatuses based on the countermeasure information using environment control estimated by the environment control estimation unit 221 (Step S305).

Next, the notification unit 240 notifies a nurse or the like about the countermeasure information other than that using environment control (Step S306). The nurse or the like can appropriately deal with the target patient based on the countermeasure information other than that using environment control notified from the notification unit 240. Note the notification unit 240 may notify the nurse or the like about environment control information estimated by the environment control estimation unit 221. By doing the above, the nurse or the like can confirm whether or not the environment management system according to this example embodiment is operating properly.

After Step S306 or when the value of the identification information is less than the predetermined value in Step S303 (“NO” in Step S303), the environment management system 200 determines whether a condition for ending the process is satisfied. The conditions for ending the process include a case in which the restlessness score of the target patient falls below a threshold and a case in which a set period of time has elapsed. When it is desired to operate the environment management system only at a specific time, for example, only at night when there are few nurses or the like in a hospital etc., a night shift time (e.g., 17:00 to 8:00 on the next day) or a lights-out time (e.g., 21:00 to 6:00 on the next day) may be set. If the condition for ending the process is satisfied (“YES” in Step S307), the operation of the environment management system 200 is ended. On the other hand, if the condition for ending the process is not satisfied (“NO” in Step S307), the environment management system 200 returns to Step S301.

The flowchart of FIG. 8 shows a specific order of execution of the operations. However, the order of execution of the operations may be different from that shown in FIG. 8. For example, the order of execution of two or more steps may be the reverse of the order shown in FIG. 8. Further, two or more consecutive steps shown in FIG. 8 may be performed simultaneously or some of these steps may be performed simultaneously. Furthermore, in some example embodiments, one or a plurality of steps shown in FIG. 8 may be skipped or omitted.

### Third Example Embodiment

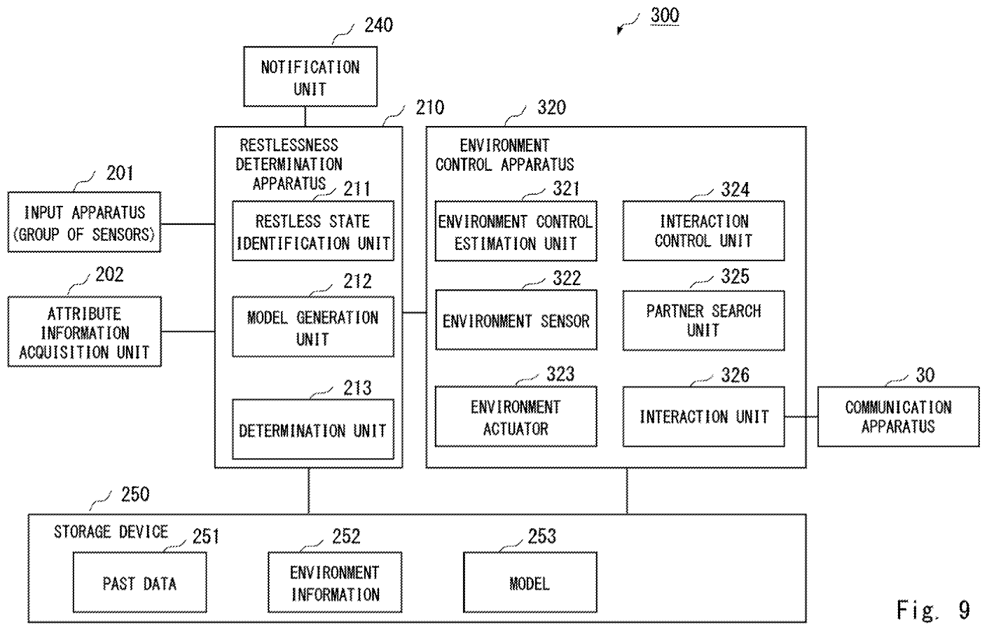

FIG. 9 is a block diagram showing a configuration of an environment management system according to a third example embodiment of the present invention. In FIG. 9, the same components as those in the second example embodiment are denoted by the same reference numerals as those of FIG. 5, and the descriptions thereof will be omitted as appropriate. In this example embodiment, an environment control apparatus 320 includes an environment control estimation unit 321, an environment sensor 322, an environment actuator 323, an interaction control unit 324, a partner search unit 325, and an interaction unit 326.

The interaction control unit 324 operates an interaction program when the determination unit 213 (the restlessness determination apparatus 210) determines that a target patient is in a restless state. Specifically, 1) when a restlessness score θ becomes a threshold T1<θ, and 2) when the score θ remains at T2<θ for a fixed period of time U2, the interaction control unit 324 operates the interaction program.

The interaction program is a program for allowing a target patient to interact with another person (e.g., another patient, a family member, a care staff such as a nurse, and a virtual person) via a communication apparatus. The interaction is, for example, one in which a target patient and another person are made to have a conversation through a communication apparatus (e.g., a smartphone and a wireless network), they show their images to each other, or they are made to play a game which matches the target patient against the other person. The communication apparatus is not limited to a smartphone, and may be, for example, a cellular phone, a personal computer, a smartwatch, a smart speaker, a projector, a camera, a television set, or a suitable combination thereof.

As described above, the partner search unit 325 searches for a partner (another person) who interacts with a target patient. The partner search unit 325 may search for an available partner from a list of people (e.g., family members, friends, and nurses) who are close to the target patient registered in advance. Alternatively, the partner search unit 325 may search for a patient who is awake among a plurality of other patients who are wearing the input apparatus 201 etc. like the target patient. The partner (the other person) who interacts with the target patient may be another patient in the same facility (hospital), a family member or close person in a remote place, or another patient in another hospital. Further, either one partner (one other person) or a plurality of partners (a plurality of other persons) may interact with the target patient. The target patient interacts with the partner who has been searched for in this manner via the communication apparatus, whereby the restless state of the target patient can be relieved.

The partner (the other person) who interacts with the target patient has a communication apparatus 30 (e.g., a smartphone and a wireless network) like the one the target patient has.

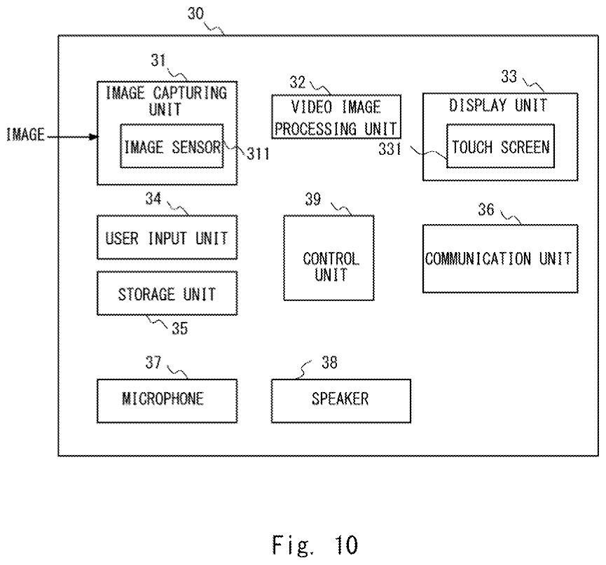

FIG. 10 is a block diagram showing an example of a configuration of the communication apparatus. As shown in FIG. 10, the communication apparatus 30 includes an image capturing unit (e.g., a camera) 31 that captures an external video image, a video image processing unit 32 that processes a video image signal (including a three-dimensional model video image signal) received from the outside, a display unit 33 that displays a video image signal processed by the video image processing unit 32 as a video image, an input unit 34 that receives an input from a user, a storage unit 35 that stores various types of data, a communication unit 36 that performs wired and wireless communication with the outside, a microphone 37, a speaker 38, and a control unit 39 that controls each component of the communication apparatus 30. The image capturing unit (e.g., a camera) 31 includes an image sensor 311 (e.g., a CCD/CMOS image sensor). Further, the display unit 33 includes a touch screen 331. The control unit 39 includes a central processing unit (CPU), a main storage device, an auxiliary storage device, and the like.

The partner (the other person) who interacts with the target patient can interact with him/her by using the communication apparatus 30 like the target patient does.

Further, when, in particular, the partner (the other person) who interacts with the target patient is another patient managed by the environment management system according to this example embodiment like the target patient, an environment management system 300 includes, in consideration of the other patient, the input apparatus 201, the attribute information acquisition unit 202, the restlessness determination apparatus 210, the environment control apparatus 320, the notification unit 240, and the storage device 250.

The interaction unit 326 changes a display content of the display unit 33 or a voice content of the speaker 38 in accordance with the behavior of the target patient and the other person acquired by the image capturing unit 31 or the microphone 37. Similarly, the partner (the other person) who interacts with the target patient changes a display content of the display unit 33 or a voice content of the speaker 38 in accordance with the behavior of the target patient and the other person acquired by the image capturing unit 31 or the microphone 37. Specifically, when the target patient and the other person show their video images to each other, the video images are switched by the operation of the target patient and the other person. For example, characters move or the background changes as in an animation. Further, in a case in which the target patient and the other person play a game which matches them against each other, the scores of the target patient and the other person are displayed and they compete with each other to obtain the highest score. For example, when they raise their hands while lying on the bed in a timely manner and when they clench and unclench their fists in a timely manner, they can receive points, while when they protrude their hands and feet from the bed and when they make a sound, their points are reduced. By doing the above, the environment of the target patient can be changed by the interaction between the target patient and the other person, and thus the attention of the target patient can be continuously attracted. Consequently, the effect of suppressing restlessness can be maintained.

The interaction unit 326 includes an indicator that indicates amounts like those shown in a bar graph or a pie graph, and operates to change a display so that the amounts increase or decrease when a specific behavior of a target patient or a specific behavior of another person is detected. By doing the above, it is easy to attract the attention of the target patient and it is possible to prevent the target patient from being bored since a sense of achievement is further given to him/her. Since the element of competition (a game) with another person is added, it is possible for this reason also to prevent the target patient from being bored. The interaction unit 326 may change the environment so that it does not respond to large movements of the bodies of the patient and the other person or their loud voices. By doing the above, it is possible to prevent the target patient from being excessively excited.

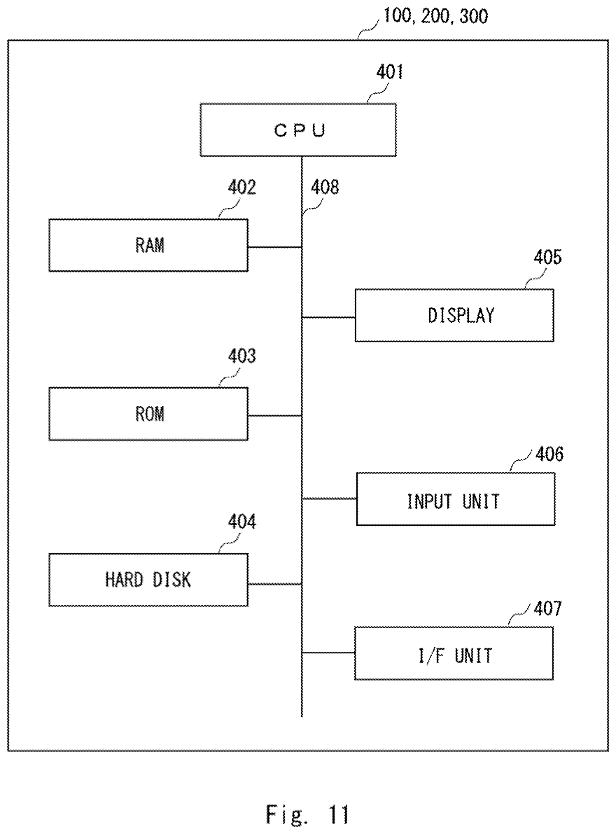

FIG. 11 is a block diagram showing an example of a hardware configuration of each of the environment management systems 100, 200, and 300. As shown in FIG. 11, each of the environment management systems 100, 200, and 300 is a computer including a Central Processing Unit (CPU) 401, a Random Access Memory (RAM) 402, and a Read Only Memory (ROM) 403. The CPU 401 performs calculation and control in accordance with software stored in the RAM 402, the ROM 403, or a hard disk 404. The RAM 402 is used as a temporary storage area when the CPU 401 executes various types of processing. The hard disk 404 stores an operating system (OS), a registration program which will be described later, and the like. A display 405 is composed of a liquid crystal display and a graphic controller, and the display 405 displays objects such as images and icons, a GUI, and the like. An input unit 406 is an apparatus for a user to give various instructions to the environment management system, and is composed of, for example, a mouse and a keyboard. An I/F (interface) unit 207 can control wireless LAN communication and wired LAN communication conforming to standards such as IEEE 802.11a, and communicates with an external device through a communication network the same as the wireless and wired LAN communication networks and the Internet based on a protocol such as TCP/IP. A system bus 408 controls the exchange of data with the CPU 401, the RAM 402, the ROM 403, the hard disk 404, and the like.

In the above-described examples, the program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (Read Only Memory), CD-R, CD-R/W, DVD (Digital Versatile Disc), BD (Blu-ray (Registered Trademark) Disc), and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

Note that the present invention is not limited to the above-described example embodiments and may be changed as appropriate without departing from the spirit of the present invention. A plurality of examples described above may be appropriately combined with one another.

Although the present invention has been described with reference to the example embodiments, the present invention is not limited to the above-described example embodiments. Various changes that may be understood by those skilled in the art may be made to the configurations and details of the present invention within the scope of the invention.

The whole or part of the example embodiments disclosed above can be described as, but not limited to, the following supplementary notes.

(Supplementary Note 1)

An environment management system comprising:

a determination unit configured to determine, based on a feature value of biological information of a target patient to be input, identification information indicating a condition of the target patient;

an estimation unit configured to estimate environment control information for the target patient based on the identification information and an environment control prediction parameter that has been learned in advance; and

an environment control unit configured to automatically change a surrounding environment of the target patient based on the environment control information for the target patient estimated by the estimation unit.

(Supplementary Note 2)

The environment management system according to Supplementary note 1, further comprising a sensor configured to continuously acquire time series data of the biological information of the target patient.

(Supplementary Note 3)

The environment management system according to Supplementary note 2, wherein

the determination unit continuously determines the identification information based on feature values of the time series data of the biological information of the target patient acquired by the sensor, and

when the determination unit determines that the condition of the target patient is a normal state, the environment control unit ends the changing of the surrounding environment of the target patient.

(Supplementary Note 4)

The environment management system according to any one of Supplementary notes 1 to 3, wherein the estimation unit estimates countermeasure information other than that using environment control in addition to the environment control information.

(Supplementary Note 5)

The environment management system according to Supplementary note 4, further comprising a notification unit configured to send a notification about the countermeasure information other than that using environment control.

(Supplementary Note 6)

The environment management system according to any one of Supplementary notes 1 to 5, wherein the environment control unit is configured to change an environment so that a sensory organ of the target patient is stimulated.

(Supplementary Note 7)

The environment management system according to any one of Supplementary notes 1 to 6, wherein the environment control unit operates an interaction program for allowing the target patient to interact with another person, and automatically changes the surrounding environment of the target patient in accordance with behavior of the target patient and behavior of the other person.

(Supplementary Note 8)

The environment management system according to Supplementary note 7, further comprising a partner search unit configured to search for, as the other person, another patient who is awake.

(Supplementary Note 9)

An environment management method comprising:

determining, based on a feature value of biological information of a target patient to be input, identification information indicating a condition of the target patient;

estimating environment control information for the target patient based on the identification information and an environment control prediction parameter that has been learned in advance; and

automatically changing a surrounding environment of the target patient based on the estimated environment control information for the target patient.

(Supplementary Note 10)

A program for causing a computer to execute:

processing of determining, based on a feature value of biological information of a target patient to be input, identification information indicating a condition of the target patient;

processing of estimating environment control information for the target patient based on the identification information and an environment control prediction parameter that has been learned in advance; and

processing of automatically changing a surrounding environment of the target patient based on the estimated environment control information for the target patient.

This application is based upon and claims the benefit of priority from Japanese patent application No. 2019-213530, filed on Nov. 26, 2019, the disclosure of which is incorporated herein in its entirety by reference.

### REFERENCE SIGNS LIST

100 ENVIRONMENT MANAGEMENT SYSTEM
110 DETERMINATION UNIT
120 ENVIRONMENT CONTROL ESTIMATION UNIT
130 ENVIRONMENT CONTROL UNIT
200 ENVIRONMENT MANAGEMENT SYSTEM
201 INPUT APPARATUS
202 ATTRIBUTE INFORMATION ACQUISITION UNIT
210 RESTLESSNESS DETERMINATION APPARATUS
211 RESTLESS STATE IDENTIFICATION UNIT
212 MODEL GENERATION UNIT
2121 IDENTIFICATION PARAMETER LEARNING UNIT
2122 ENVIRONMENT CONTROL PREDICTION PARAMETER LEARNING UNIT
213 DETERMINATION UNIT
220 ENVIRONMENT CONTROL APPARATUS
221 ENVIRONMENT CONTROL ESTIMATION UNIT
222 ENVIRONMENT SENSOR
223 ENVIRONMENT ACTUATOR
240 NOTIFICATION UNIT
250 STORAGE DEVICE
251 PAST DATA
252 ENVIRONMENT INFORMATION
253 MODEL
300 ENVIRONMENT MANAGEMENT SYSTEM
320 ENVIRONMENT CONTROL APPARATUS
321 ENVIRONMENT CONTROL ESTIMATION UNIT
322 ENVIRONMENT SENSOR
323 ENVIRONMENT ACTUATOR
324 INTERACTION CONTROL UNIT
325 PARTNER SEARCH UNIT
326 INTERACTION UNIT

What is claimed is:

1. An environment management system comprising:
determination unit configured to determine, based on a feature value of biological information of a target patient to be input, identification information indicating a condition of the target patient;
estimation unit configured to estimate environment control information for the target patient based on the identification information and an environment control prediction parameter that has been learned in advance; and
environment control unit configured to automatically change a surrounding environment of the target patient based on the environment control information for the target patient estimated by the estimation unit.

2. The environment management system according to claim 1, further comprising a sensor configured to continuously acquire time series data of the biological information of the target patient.

3. The environment management system according to claim 2, wherein
the determination unit continuously determines the identification information based on feature values of the time series data of the biological information of the target patient acquired by the sensor, and
when the determination unit determines that the condition of the target patient is a normal state, the environment control unit ends the changing of the surrounding environment of the target patient.

4. The environment management system according to claim 1, wherein the estimation unit estimates countermeasure information other than that using environment control in addition to the environment control information.

5. The environment management system according to claim 4, further comprising notification unit configured to send a notification about the countermeasure information other than that using environment control.

6. The environment management system according to claim 1, wherein the environment control unit is configured to change an environment so that a sensory organ of the target patient is stimulated.

7. The environment management system according to claim 1, wherein the environment control unit operates an interaction program for allowing the target patient to interact with another person, and automatically changes the surrounding environment of the target patient in accordance with behavior of the target patient and behavior of the other person.

8. The environment management system according to claim 7, further comprising partner search unit configured to search for, as the other person, another patient who is awake.

9. An environment management method comprising:
determining, based on a feature value of biological information of a target patient to be input, identification information indicating a condition of the target patient;
estimating environment control information for the target patient based on the identification information and an environment control prediction parameter that has been learned in advance; and
automatically changing a surrounding environment of the target patient based on the estimated environment control information for the target patient.

10. A non-transitory computer readable medium storing a program for causing a computer to execute:
processing of determining, based on a feature value of biological information of a target patient to be input, identification information indicating a condition of the target patient;
processing of estimating environment control information for the target patient based on the identification information and an environment control prediction parameter that has been learned in advance; and
processing of automatically changing a surrounding environment of the target patient based on the estimated environment control information for the target patient.
